(19) United States
(12) Patent Application Publication
(10) Pub. No.: US 2021/0088523 A1
(43) Pub. Date: Mar. 25, 2021

(54) METHODS FOR DETECTING CIRCULATING TUMOR CELLS IN NON-SMALL CELL LUNG CANCER

(71) Applicant: SRI INTERNATIONAL, Menlo Park, CA (US)

(72) Inventors: Xiaohe Liu, Palo Alto, CA (US); Lidia C. Sambucetti, Redwood City, CA (US); Peter B. Madrid, San Jose, CA (US); Zheng Ao, Bloomington, IN (US)

(21) Appl. No.: 16/838,651

(22) Filed: Apr. 2, 2020

Related U.S. Application Data
(60) Provisional application No. 62/904,059, filed on Sep. 23, 2019.

(51) Int. Cl. G01N 33/574; G01N 33/58

(52) U.S. Cl. CPC G01N 33/57492; G01N 33/58

(57) ABSTRACT

Disclosed herein are methods of detecting circulating tumor cells by detecting one or more of pan-cytokeratin, epidermal growth factor receptor, human epidermal growth factor receptor 2, mucin 1, plastin 3, circulating cancer associated fibroblast and programmed death-ligand using fiber-optic array scanning technology and automated digital microscopy. The methods disclosed herein can also be used for cancer diagnosis, prognosis, and in the design of cancer treatments.

### CROSS-REFERENCE TO RELATED APPLICATIONS

This application claims the benefit of U.S. Provisional Application No. 62/904,059, filed on Sep. 23, 2019. The content of this earlier filed application is hereby incorporated by reference herein in its entirety.

### BACKGROUND

Metastasis accounts for >90% of cancer related mortality. For non-small cell lung cancer (NSCLC), patients with distant metastatic tumors has about 10-fold lower 5 year survival rate (6%) as compared with those diagnosed at localized stage (60%) (Howlader, N., et al., SEER cancer statistics review, 1975-2012. Bethesda, Md.: National Cancer Institute, 2015). Thus, it is important to develop tests to diagnose metastatic lung cancer as well as provide longitudinal monitoring of the disease. Currently, NSCLC was mostly diagnosed and evaluated at follow-up visits with computed tomography (CT) scans. Technologies such as low dose CT scans bring hope for early lung cancer detection, which was shown in clinical studies to reduce mortality rate as much as 20% (Team, N.L.S.T.R., New England Journal of Medicine, 2011. 365(5): p. 395-409). However, the challenges for lung cancer diagnostics still exist since CT scans require expensive equipment set-up and can expose patients to harmful radiation thus cannot be repeated often.

### SUMMARY

Disclosed herein are methods of detecting one or more circulating tumor cells, the methods comprising: obtaining or having obtained nucleated cells from a subject; contacting the nucleated cells with an anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody and an anti-plastin 3 (PLS3) antibody; and detecting binding of the anti-pan-CK antibody, the anti-EGFR antibody, the anti-HER2 antibody, the anti-MUC1 antibody and the anti-PLS3 antibody to the nucleated cells, wherein binding of the anti-pan-CK antibody, the anti-EGFR antibody, the anti-HER2 antibody, the anti-MUC1 antibody and the anti-PLS3 antibody to the nucleated cells detects one or more circulating tumor cells.

Disclosed herein are methods of diagnosing non-small cell lung cancer in a subject, the methods comprising: obtaining or having obtained nucleated cells from a subject; contacting the nucleated cells with a slide comprising a surface coated with antibodies that bind one or more cell surface markers pan-cytokeratin (pan-CK), epidermal growth factor receptor (EGFR), human epidermal growth factor receptor 2 (HER2), mucin 1 (MUC1) and plastin 3 (PLS3); detecting the presence of immunostaining of the nucleated cells with the antibodies by scanning the slide using fiber-optic array scanning technology (FAST), wherein a cell positive for pan-CK, EGFR, HER2 or PLS3 is a circulating tumor cell; and identifying the subject as having non-small-cell lung cancer based on the presence of the circulating tumor cell that expresses the one or more cell surface markers in the sample.

Disclosed herein are methods of detecting and treating non-small-cell lung cancer in a subject, the methods comprising: obtaining or having obtained nucleated cells from a subject; contacting the nucleated cells with an anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody, an anti-plastin 3 (PLS3) antibody, and an anti-programmed death-ligand (PD-L1) antibody; detecting binding of the anti-pan-CK antibody, the anti-EGFR antibody, the anti-HER2 antibody, the anti-MUC1 antibody, the anti-PLS3 antibody, and the anti-PD-L1 antibody to the nucleated cells, wherein binding of the anti-pan-CK antibody, the anti-EGFR antibody, the anti-HER2 antibody, the anti-MUC1 antibody, the anti-PLS3 antibody and the anti-PD-L1 antibody is to the nucleated cells, wherein a cell positive for pan-CK, EGFR, HER2, MUC1, PLS3, and PD-L1 indicates non-small-cell lung cancer in the subject; and treating the non-small-cell lung cancer in the subject with a PD-L1 inhibitor.

### BRIEF DESCRIPTION OF THE DRAWINGS

FIGS. 1A-B show a tumor marker cocktail to detect NSCLC CTC. FIG. 1A shows a tumor marker cocktail optimization on A549, H222, H522 cell lines. FIG. 1B shows a comparison of CTC number detected in NSCLC patient blood using cytokeratin or a tumor marker cocktail. The tumor marker cocktail comprises an anti-pan-CK antibody, an anti-EGFR antibody, an anti-HER2 antibody, an anti-MUC1 antibody and an anti-PLS3 antibody.

FIGS. 2A-C show the optimization and characterization of mesenchymal CTC. FIG. 2A shows three NSCLC cell lines used to optimize Vimentin staining: A549, H2228 and H522. They range from epithelial to mesenchymal phenotype. Cells were stained as DAPI (blue), cytokeratin (red), vimentin (Green). FIG. 2B shows Vimentin positive CTC was identified in NSCLC patients using cocktail detection (Left) and cocktail detection of CTC (Right). Detected CTC was shown as Vimentin positive EMT CTC. FIG. 2C shows Vimentin positive and vimentin negative CTC frequency in NSCLC blood sample analyzed by CK or cocktail. The cocktail comprises an anti-pan-CK antibody, an anti-EGFR antibody, an anti-HER2 antibody, an anti-MUC1 antibody and an anti-PLS3 antibody.

FIGS. 3A-E show detection of cCAF in NSCLC patients using FAST platform. FIG. 3A shows detection of CAF21 cells spiked in blood. CAF21 cells were stained with anti-FAP (Red) and leukocytes were stained with anti-CD45 (Green). FIG. 3B shows detection sensitivity and accuracy of FAST platform. 10, 25 and 50 CAF-21 cells were spiked in 10 mL of blood and enumerated by CAF. FIGS. 3C and 3D show immunofluorescence staining of cCAF detected in NSCLC (C: FAP Red, CD45 Green. D: FAP Red, CK Green). FIG. 3E show detection sensitivity of cCAF, CTC or combined detection in NSCLC.

FIGS. 4A-C show PDL expression in NSCLC cell lines. FIG. 4A shows PD-L1 (Green) staining on high, medium and low expressing NSCLC cell lines (CK, red). FIG. 4B shows PD-L1 expression level analysis on NSCLC cell lines. FIG. 4C shows CTC identified by antibody cocktail showed various PD-L1 positivity in NSCLC patients. The antibody cocktail comprises an anti-pan-CK antibody, an anti-EGFR antibody, an anti-HER2 antibody, an anti-MUC1 antibody and an anti-PLS3 antibody.

FIG. 5 is a table showing PD-L1 expression analysis on CTC.

FIG. 6 is a table showing a comparison of CTC detection using CK versus the disclosed cocktail, comprising an anti-pan-CK antibody, an anti-EGFR antibody, an anti-HER2 antibody, an anti-MUC1 antibody and an anti-PLS3 antibody.

FIG. 7 is a table showing Vimentin+CTC detected by CK and the disclosed cocktail, comprising an anti-pan-CK antibody, an anti-EGFR antibody, an anti-HER2 antibody, an anti-MUC1 antibody and an anti-PLS3 antibody.

### DETAILED DESCRIPTION

The disclosed method and compositions may be understood more readily by reference to the following detailed description of particular embodiments and the Examples included therein and to the Figures and their previous and following description.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”, meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for non-small cell lung cancer, such as, for example, prior to the administering step.

As used herein, “sample” is meant to mean an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile, broncheoalveolar lavage fluid, pleural fluid, fine needle aspirate, tissue, whole lung lavage fluid, pulmonary vein blood, cerebrospinal fluid) that contains cells or cell components.

“Inhibit,” “inhibiting,” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100 percent, or any amount of reduction in between as compared to native or control levels. In an aspect, the inhibition or reduction is 10-20, 20-30, 30-40, 40-50, 50-60, 60-70, 70-80, 80-90, or 90-100 percent as compared to native or control levels. In some aspects, the inhibition or reduction is 0-25, 25-50, 50-75, or 75-100 percent as compared to native or control levels.

The term “contacting” as used herein refers to bringing an antibody, a capture agent, compound or test agent and a cell, target receptor, antigen, peptide, protein, or other biological entity together in such a manner that the an antibody, a capture agent, compound or test agent can interact with the cell, target receptor, antigen, peptide, protein, or other biological entity (e.g., by interacting with the cell, target receptor, antigen, peptide, protein, or other biological entity).

As used herein, the term “level” refers to the amount of a target molecule (e.g., cell surface marker-expressing cell) in a sample, e.g., a sample from a subject. The amount of the cell target molecule (e.g., surface marker-expressing cell) can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compares to a control (i.e., a non-affected or healthy subject or a sample from a non-affected or healthy subject) or a sham or an untreated sample) or comparing two or more samples obtained from the same subject but at different time points.

The phrase “at least” preceding a series of elements is to be understood to refer to every element in the series. For example, “at least one” includes one, two, three, four or more.

As used herein, “effective amount” of a compound is meant to mean a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

As used herein, “treat” is meant to mean administer a compound or molecule of the invention to a subject, such as a human or other mammal (for example, an animal model), that has a cancer, in order to prevent or delay a worsening of the effects of the disease or condition, or to partially or fully reverse the effects of the disease.

As used herein, “prevent” is meant to mean minimize the chance that a subject who has an increased susceptibility for developing cancer will develop cancer. As further used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “reference,” “reference expression,” “reference sample,” “reference value,” “control,” “control sample” and the like, when used in the context of a sample or level or amount of cell surface marker-expressing cells refers to a reference standard wherein the reference is expressed at a constant level and is unaffected by the experimental conditions, and is indicative of the level in a sample of a predetermined disease status (e.g., not suffering from a cancer or metastastis). The reference value can be a predetermined standard value or a range of predetermined standard values, representing no illness, or a predetermined type or severity of illness.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to an examination by a person of skill, for example, a physician, and found to have a condition that can be diagnosed or treated by a compound or composition disclosed herein.

As used herein, “specifically binds” is meant that an antibody recognizes and physically interacts with its cognate antigen or target and does not significantly recognize and interact with other antigens or targets; such an antibody may be a polyclonal antibody or a monoclonal antibody, which are generated by techniques that are well known in the art.

As described herein, the term “immunologically binding” is a non-covalent form of attachment between an epitope of an antigen (e.g., the epitope-tag) and the antigen-specific part of an antibody or fragment thereof. Antibodies are preferably monoclonal and must be specific for the respective epitope tag(s) as used. Antibodies include murine, human and humanized antibodies. Antibody fragments are known to the person of skill and include, amongst others, single chain Fv antibody fragments (scFv fragments) and Fab-fragments. The antibodies can be produced by regular hybridoma and/or other recombinant techniques. Many antibodies are commercially available.

As used herein, “probe,” “primer,” or “oligonucleotide” is meant to mean a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

As used herein, “specifically hybridizes” is meant to mean that a probe, primer, or oligonucleotide recognizes and physically interacts (that is, base-pairs) with a substantially complementary nucleic acid under high stringency conditions, and does not substantially base pair with other nucleic acids.

As used herein, “high stringency conditions” is meant to mean conditions that allow hybridization comparable with that resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65° C., or a buffer containing 48% formamide, 4.8×SSC, 0.2 M Tris-C1, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

As used herein, a “control” is a sample from either a normal subject or a sample that is not cancerous.

The term “antibody” as used herein is intended to include, without limitation, whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker, or mixtures thereof. The scFv's may be covalently or non-covalently linked to form antibodies having two or more binding sites. Polyclonal, monoclonal, or other purified preparations of antibodies and recombinant antibodies may be incorporated or used with the example embodiments. An antibody used for detection of a biomarker or cell surface receptor, as described herein, may be a labeled antibody. For example, an antibody or labeled antibody may comprise a fluorescent label for detection and/or capture of circulating tumor cell surface or cytosolic markers selected, without limitation, from the group consisting of pan-CK, EGFR, HER2, Mucin-1, PLS3, PD-L1 and FAP (as a marker for circulating cancer associated fibroblasts (cCAF)), as further described herein.

As used herein, the term “prognosis” defines a forecast as to the probable outcome of a disease, the prospect as to recovery from a disease, or the potential recurrence of a disease as indicated by the nature and symptoms of the case.

The term “circulating tumor cell” (CTC) is intended to mean any cancer cell that is found in a subject's sample. Typically, CTCs are exfoliated from a solid tumor. As used herein, the term “circulating cells” comprises extratumoral cells that have either metastasized or micrometastasized from a solid tumor. Examples of circulating cells include, but are not limited to, circulating tumor cells, cancer stem cells, and/or cells that are migrating to the tumor (e.g., circulating cancer associated fibroblasts (cCAFs), circulating endothelial progenitor cells, circulating endothelial cells, circulating pro-angiogenic myeloid cells, circulating dendritic cells, etc.). Patient samples containing circulating cells can be obtained from any accessible biological fluid (e.g., whole blood, serum, plasma, sputum, bronchial lavage fluid, urine, nipple aspirate, lymph, saliva, fine needle aspirate, etc.). In some aspects, the whole blood sample is separated into a plasma or serum fraction and a cellular fraction (i.e., cell pellet). The cellular fraction typically contains red blood cells, white blood cells, and/or circulating cells of a solid tumor such as circulating tumor cells (CTCs), circulating cancer associated fibroblasts (cCAFs), circulating endothelial cells (CECs), circulating endothelial progenitor cells (CEPCs), cancer stem cells (CSCs), disseminated tumor cells of the lymph node, and combinations thereof. The plasma or serum fraction usually contains, inter alia, nucleic acids (e.g., DNA, RNA) and proteins that are released by circulating cells of a solid tumor.

Circulating tumor cells are typically isolated from a patient sample using one or more separation methods including, for example, immunomagnetic separation (see, e.g., Racila et al., Proc. Natl. Acad. Sci. USA, 95:4589-4594 (1998); Bilkenroth et al., Int. J. Cancer, 92:577-582 (2001)), the CellTracks® System by Immunicon (Huntingdon Valley, Pa.), microfluidic separation (see, e.g., Mohamed et al., IEEE Trans. Nanobiosci., 3:251-256 (2004); Lin et al., Abstract No. 5147, 97th AACR Annual Meeting, Washington, D.C. (2006)), FACS (see, e.g., Mancuso et al., Blood, 97:3658-3661 (2001)), density gradient centrifugation (see, e.g., Baker et al., Clin. Cancer Res., 13:4865-4871 (2003)), and depletion methods (see, e.g., Meye et al., Int. J. Oncol., 21:521-530 (2002)).

As used herein, the term “cell” or “cells” refers to a single cell as well as a plurality or population of cells.

As used herein, the term “marker” or “biomarker” or “cell-surface marker” refers to a biological molecule, e.g., a nucleic acid, peptide, protein, hormone, etc., whose presence or concentration can be detected and correlated with a known condition, such as a disease state. Examples of markers, biomarkers or cell-surface markers include but are not limited to pan-CK, EGFR, HER2, Mucin-1, PLS3, cCAG and PD-L1.

A “biopsy” refers to the process of removing a tissue sample for diagnostic or prognostic evaluation, and to the tissue specimen itself. Any biopsy technique known in the art can be applied to the methods and compositions of the present invention. The biopsy technique applied will generally depend on the tissue type to be evaluated and the size and type of the tumor (i.e., solid or suspended (i.e., blood or ascites)), among other factors. Representative biopsy techniques include excisional biopsy, incisional biopsy, needle biopsy (e.g., core needle biopsy, fine-needle aspiration biopsy, etc.), surgical biopsy, and bone marrow biopsy. Biopsy techniques are discussed, for example, in Harrison's Principles of Internal Medicine, Kasper, et al., eds., 16th ed., 2005, Chapter 70, and throughout Part V. One skilled in the art will appreciate that biopsy techniques can be performed to identify cancerous and/or precancerous cells in a given tissue sample. In some aspects, the biopsy can be a liquid biopsy.

The term “incubating” is used synonymously with “contacting” and “exposing” and does not imply any specific time or temperature requirements unless otherwise indicated.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Also disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein.

Liquid biopsy tests aim at detecting cancer cells (CTC) and DNA (ctDNA) through a simple blood draw. It has the benefit of being minimally invasive with possibility of repeatable sampling and relatively low cost (Alix-Panabieres, C. and K. Pantel, Clinical chemistry, 2013. 59(1): p. 110-118; Alix-Panabieres, C. and K. Pantel, Cancer discovery, 2016. 6(5): p. 479-491; Cayrefourcq, L. and C. Alix-Panabieres, 2019, IntechOpen; and Pantel, K. and C. Alix-Panabieres, Nature Reviews Clinical Oncology, 2019. 16(7): p. 409-424). Circulating tumor DNA can be detected by deep sequencing in plasma and is easily amendable to standardized assays (Jiang, T., S. Ren, and C. Zhou, Lung Cancer, 2015. 90(2): p. 128-34; Thress, K. S., et al., Lung cancer, 2015. 90(3): p. 509-515; and Abbosh, C., et al., Nature, 2017. 545(7655): p. 446-451). However, recent studies have shown that ctDNA may suffer from high false positive detection in healthy controls and has limited clinical utility in early stage cancer detection (Merker, J. D., et al., J Clin Oncol, 2018. 36(16): p. 1631-1641; and Razavi, P., et al., Nature Medicine, 2019. 25(12): p. 1928-1937). In contrast, CTC tests have the advantage of producing very low false positive cases (Hou, J.-M., et al., J Clin Oncol, 2012. 30(5): p. 525-532; Krebs, M. G., et al., J Clin Oncol, 2011. 29(12): p. 1556-1563; Zhou, M.-D., et al., Scientific Reports, 2014. 4(1): p. 7392; Williams, A., et al., Cancer Research, 2012. 72(8 Supplement): p. 2372-2372; and Miyamoto, D. T., et al., Cancer Discovery, 2018. 8(3): p. 288-303). And preliminary studies have shown that CTC number is an independent predictive marker for recurrent lung cancer relapse (Hofman, V., et al., Clin Cancer Res, 2011. 17(4): p. 827-35; and Hofman, V., et al., Int J Cancer, 2011. 129(7): p. 1651-60). Moreover, recent studies interrogating pulmonary vein CTC (PV-CTC) also indicated that PV-CTC can reveal metastatic mutations as early as 10 months before recurrence (Chemi, F., et al., Nat Med, 2019. 25(10): p. 1534-1539). Despite the promising utility of CTC in lung cancer, the detection sensitivity remains an issue that hinders CTC's application in NSCLC. In the past, CTC tests such as FDA-cleared CellSearch has a reported sensitivity of 27% in detecting NSCLC (Hofman, V., et al., Clin Cancer Res, 2011. 17(4): p. 827-35). This low sensitivity is partially due to the fact that most CTC, especially CTC in NSCLC underwent a process known as epithelial-mesenchymal transition (EMT). During EMT, CTC will shed off epithelial marker expression such as Epithelial cell adhesion molecule (EpCAM) (Yu, M., et al., Science (New York, N.Y.), 2013. 339(6119): p. 580-584). Thus, these mesenchymal CTC will become “invisible” to the technologies relying on the expression of these markers such as CellSearch technology (Yu, M., et al., Science (New York, N.Y.), 2013. 339(6119): p. 580-584). In contrast, antigen agnostic separations, such as microfilter or microfluidics-based technologies, were shown to detect mesenchymal CTC and increase CTC detection rate (Zheng, S., et al., Journal of Chromatography A, 2007. 1162(2): p. 154-161; Lin, H. K., et al., Clinical Cancer Research, 2010. 16(20): p. 5011-5018; Zheng, S., et al., Biomedical Microdevices, 2011. 13(1): p. 203-213; Huang, Q., et al., Theranostics, 2018. 8(6): p. 1624-1635; Ao, Z., et al., R. J. Cote and R. H. Datar, Editors. 2016, Springer New York: New York, N.Y. p. 29-45; Cheng, Y.-H., et al., Nature Communications, 2019. 10(1): p. 2163; Edd, J. F., et al., Lab on a Chip, 2020. 20(3): p. 558-567; and Dhar, M., et al., Proceedings of the National Academy of Sciences, 2018. 115(40): p. 9986-9991). However, the problem still remains for antigen agnostic platforms to develop a comprehensive marker panel to effectively detect all tumor derived CTCs.

In addition to detection of NSCLC, CTCs also hold promise for companion diagnostics of cancer therapy. Recently, cancer immunotherapy, including immune checkpoint inhibitor (ICI) therapy has shown promising results in NSCLC (Herbst, R. S., et al., The Lancet, 2016. 387(10027): p. 1540-1550). And it has become standard of care now for many patients (Zimmermann, S., et al., Am Soc Clin Oncol Educ Book, 2018. 38: p. 682-695). Currently, PD-L1 staining of primary tumor tissue is used as standard of care for ICI companion diagnostics. Ventana SP263 assay is one of the FDA-approved companion diagnostic (cDx) tests for primary tissue PD-L1 staining (Ancevski Hunter, K., M. A. Socinski, and L. C. Villaruz, Molecular diagnosis & therapy, 2018. 22(1): p. 1-10). It was shown that patients with PD-L1+ tumors have higher (27%) objective response rates (ORR) as compared with patients with PD-L1− tumors (5%) (Hui, R., et al., Ann Oncol, 2017. 28(4): p. 874-881). However, primary tumor staining requires surgery, and can be sampled just once. CTC tests, benefiting from its simplicity and minimal invasiveness as well as feasibility for real-time re-sampling, serve as an excellent candidate for ICI treatment companion diagnostics (Anantharaman, A., et al., BMC Cancer, 2016. 16(1): p. 744). In fact, PD-L1 expression on CTC was shown to correlate poor survival in breast cancer patients (Wang, X., G. Zhang, and Q. Sun, PD-L1 expression on circulating tumor cells and prognosis of breast cancer patients. Journal of Clinical Oncology, 2019. 37(15 suppl): p. e14028-e14028). In NSCLC, CTC expression of PD-L1 was shown to increase as disease progresses (Chen, Y. L., et al., Cancer Immunol Immunother, 2019. 68(7): p. 1087-1094; Adams, D. L., et al., Clin Cancer Res, 2017. 23(19): p. 5948-5958; Wang, Y., et al., Scientific Reports, 2019. 9(1): p. 566; and Janning, M., et al., 2019. 11(6): p. 835).

### Methods

Disclosed herein methods of detecting one or more circulating tumor cells. In some aspects, the methods can comprise the step of obtaining or having obtained nucleated cells from a subject. In some aspects, the methods can comprise contacting the nucleated cells with one or more of an anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody and an anti-plastin 3 (PLS3) antibody. In some aspects, the methods can comprise contacting the nucleated cells with an anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody and an anti-plastin 3 (PLS3) antibody. In some aspects, the methods can comprise detecting binding of an anti-pan-CK antibody, an anti-EGFR antibody, an anti-HER2 antibody, an anti-MUC1 antibody and/or an anti-PLS3 antibody to the nucleated cells. In some aspects, the binding of an anti-pan-CK antibody, an anti-EGFR antibody, an anti-HER2 antibody, an anti-MUC1 antibody and/or an anti-PLS3 antibody to the nucleated cells can detect one or more circulating tumor cells. In some aspects, the methods can further comprise contacting the nucleated cells with an anti-fibroblast activation protein (FAP) antibody to detect circulating cancer associated fibroblast (cCAF). In some aspects, the methods can further comprise detecting cCAF with an anti-FAP antibody. In some aspects, the methods can further comprise contacting the nucleated cells with an anti-programmed death-ligand (PD-L1) antibody. In some aspects, the methods can further comprise detecting an anti-PD-L1 antibody bound to the nucleated cells. In some aspects, the methods can further comprise contacting the nucleated cells with an anti-FAP antibody and an anti-PD-L1 antibody. In some aspects, the methods can further comprise detecting cCAF using the FAP antibody or detecting one more circulating tumor cells using the an-PD-L1 antibody. In some aspects, the binding of the FAP antibody can detect one or more cCAFs or an anti-PD-L1 antibody can detect one or more circulating tumor cells.

Disclosed herein methods of identifying one or more circulating tumor cells. In some aspects, the methods can comprise the step of obtaining or having obtained nucleated cells from a subject. In some aspects, the methods can comprise contacting the nucleated cells with one or more of an anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody and an anti-plastin 3 (PLS3) antibody. In some aspects, the methods can comprise contacting the nucleated cells with an anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody and an anti-plastin 3 (PLS3) antibody. In some aspects, the methods can comprise identifying binding of an anti-pan-CK antibody, an anti-EGFR antibody, an anti-HER2 antibody, an anti-MUC1 antibody and/or an anti-PLS3 antibody to the nucleated cells. In some aspects, the binding of an anti-pan-CK antibody, an anti-EGFR antibody, an anti-HER2 antibody, an anti-MUC1 antibody and/or an anti-PLS3 antibody to the nucleated cells can identify one or more circulating tumor cells. In some aspects, the methods can further comprise identifying one or more circulating cancer associated fibroblasts. In some aspects, the methods can further comprise detecting cCAFs using an FAP antibody. In some aspects, the methods can further comprise contacting the nucleated cells with an anti-FAP antibody. In some aspects, the methods can further comprise detecting cCAF with an anti-FAP antibody. In some aspects, the methods can further comprise contacting the nucleated cells with an anti-PD-L1 antibody. In some aspects, the methods can further comprise detecting an anti-PD-L1 antibody. In some aspects, the methods can further comprise contacting the nucleated cells with an anti-FAP antibody and an anti-PD-L1 antibody. In some aspects, the methods can further comprise detecting an FAP antibody or an anti-PD-L1 antibody. In some aspects, the binding of an anti-FAP antibody can identify one or more cCAFs or an anti-PD-L1 antibody can identify one or more circulating tumor cells. In some aspects, the circulating tumor cell can be a lung cancer cell, non-small-cell lung cancer cell, a brain cancer cell, a kidney cancer cell or a bladder cancer cell. In some aspects, the circulating tumor cell can be a metastatic cancer cell.

In some aspects, any of the methods herein can further comprise enriching the nucleated cells from the subject prior to contacting the nucleated cells with any of the antibodies disclosed herein. In some aspects, any of the methods herein can further comprise enriching the nucleated cells from the subject prior to contacting the nucleated cells with an anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody and/or an anti-plastin 3 (PLS3) antibody. In some aspects, any of the methods herein can further comprise enriching the nucleated cells from the subject prior to contacting the nucleated cells with an anti-FAP antibody and/or an anti-PD-L1 antibody. In some aspects, any of the methods disclosed herein can further comprise further comprising enriching the circulating tumor cells detected. Methods of enriching the nucleated cells are known in the art.

Disclosed herein are methods of diagnosing non-small cell lung cancer in a subject. In some aspects, the methods can include obtaining or having obtained nucleated cells from a subject. In some aspects, the methods can include contacting the nucleated cells with a slide comprising a surface coated with antibodies that bind one or more cell surface markers pan-cytokeratin (pan-CK), epidermal growth factor receptor (EGFR), human epidermal growth factor receptor 2 (HER2), mucin 1 (MUC1) and plastin 3 (PLS3). In some aspects, the methods disclosed herein can comprise detecting the presence of immunostaining of the nucleated cells with the antibodies by scanning the slide using fiber-optic array scanning technology (FAST). In some aspects, a cell positive for pan-CK, EGFR, HER2 or PLS3 can be a circulating tumor cell. In some aspects, the methods can comprise identifying the subject as having non-small-cell lung cancer based on the presence of the circulating tumor cell that expresses the one or more cell surface markers in the sample. In some aspects, the methods can further comprise providing a subsequent sample from the subject. In some aspects, the sample can be a liquid sample. In some aspects, the sample can be a blood sample. In some aspects, the sample can comprise blood from the subject. In some aspects, the method can comprise separating nucleated cells from the blood. In some aspects, the method can comprise quantifying a level of cell surface marker-expressing cells in the first and subsequent samples. In some aspects, the subsequent sample can be obtained at any time point after the first sample is obtained. In some aspects, the subsequent sample can be obtained, for example, at one or more follow-up visits, pre- and post-treatment, and, if applicable, post-mortem analysis. In some aspects, the subsequent sample can be obtained 1 day, 5 days, 10 days, 15 days, 20 days, 25 days, 30 days or any time in between after the first sample is obtained. In some aspects, the subsequent sample can be obtained 1 month, 2 months, 3 months, 3 months, 4 months, 5 months, 6 months, 7 months, 8 months, 9 months, 10 months, 11 months, 12 months after the first sample is obtained. In some aspects, the subsequent sample can be obtained 1 year, 2 years, 3 years, 4 years, 5 years, 10 years, 15 years or longer or any time in beween after the first sample is obtained. In some aspects, the increase in the number of cells that express the cell surface markers can indicate that a cancer is progressing in the subject. In some aspects, a decrease in the number of cells that express the surface markers can indicate that cancer is regressing in the subject. In some aspects, no significant change in the number of cells that express the cell surface markers can indicate that cancer is stable in the subject. In some aspects, the methods can further comprise contacting the nucleated cells with antibodies that bind to an additional cell surface marker. In some aspects, the additional cell surface marker can be FAP and/or PD-L1. In some aspects, the method can further comprise performing automated digital microscopy on the cells positive for pan-CK, EGFR, HER2, PLS3, FAP or PD-L1. In some aspects, the detecting of the immunostaining can comprise detection of the cell surface marker. In some aspects, antibodies can be used to detect the cell surface markers. In some aspects, the antibodies can be fluorescently labeled. In some aspects, the circulating tumor cell can be a non-small-cell lung cancer cell. In some aspects, the nucleated cells obtained from the subject can be from a blood sample, a peripheral blood sample, a pulmonary vein blood sample, a bronchopulmonary lavage sample, a whole lung lavage sample, a cerebral spinal fluid sample, or a urine sample.

Also disclosed herein are methods of detecting and treating non-small-cell lung cancer in a subject. In some aspects, the methods can comprise obtaining or having obtained nucleated cells from a subject. In some aspects, the methods can comprise contacting the nucleated cells with an anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody and/or an anti-plastin 3 (PLS3) antibody. In some aspects, the methods can comprise detecting binding of an anti-pan-CK antibody, an anti-EGFR antibody, an anti-HER2 antibody, an anti-MUC1 antibody and/or an anti-PLS3 antibody to the nucleated cells. In some aspects, binding of an anti-pan-CK antibody, an anti-EGFR antibody, an anti-HER2 antibody, an anti-MUC1 antibody and an anti-PLS3 antibody is to the nucleated cells. In some aspects, a cell positive for pan-CK, EGFR, HER2, PLS3, and PD-L1 can indicate non-small-cell lung cancer in the subject. In some aspects, the method can comprise treating the non-small-cell lung cancer in the subject with a PD-L1 inhibitor. In some aspects, the cell positive for PD-L1 can indicates the responsiveness of the subject to a cancer therapy. In some aspects, the cancer therapy can be a PD-L1 inhibitor. In some aspects, the PD-L1-inhibitor can be atezolizumab, durvalumab, or avelumab. In some aspects, the methods can further comprise contacting the nucleated cells with an anti-FAP antibody. In some aspects, the methods can further comprise detecting the FAP antibody. In some aspects, the contacting of the nucleated cells an anti-pan-CK antibody, an anti-EGFR antibody, an anti-HER2 antibody, an anti-MUC1 antibody, an anti-PLS3 antibody, an anti-PD-L1 antibody, and an anti-FAP antibody can be conducted on a slide. In some aspects, the detecting of the binding of an anti-pan-CK antibody, an anti-EGFR antibody, an anti-HER2 antibody, an anti-MUC1 antibody, an anti-PLS3 antibody, an anti-PD-L1 antibody, and/or an anti-FAP antibody to the nucleated cells can be conducted by scanning the slide using fiber-optic array scanning technology. In some aspects, the methods can further comprise performing automated digital microscopy on the cells positive for pan-CK, EGFR, HER2, MUC1, PLS3, FAP or PD-L1. In some aspects, an anti-pan-CK antibody, an anti-EGFR antibody, an anti-HER2 antibody, an anti-MUC1 antibody, an anti-PLS3 antibody, an anti-FAP antibody and an anti-PD-L1 can be fluorescently labeled. In some aspects, the cell positive for pan-CK, EGFR, HER2, MUC1 PLS3, FAP and/or PD-L1 can be a circulating tumor cell. In some aspects, the cell positive for FAP can be a circulating cancer-associated fibroblast. In some aspects, the circulating tumor cell can be a non-small-cell lung cancer cell. In some aspects, the nucleated cells obtained from the subject can be from a blood sample, a pulmonary vein blood sample, a bronchopulmonary lavage sample, or a whole lung lavage sample.

Sample.

In some aspects, the methods disclosed herein can include the step of obtaining or having obtained nucleated cells from a subject. In some aspects, the methods disclosed herein can include the step of obtaining or having obtained a sample from a subject. In some aspects, the nucleated cells obtained from the subject can be from any liquid biological sample. In some aspects, the nucleated cells obtained from the subject can be from a blood sample. In some aspects, the blood sample can be peripheral blood. In some aspects, the nucleated cells obtained from the subject can be from peripheral blood, pulmonary vein blood, bronchopulmonary lavage, whole lung lavage (for lung cancers), cerebral spinal fluid, and urine. In some aspects, the nucleated cells obtained from the subject can be from a blood sample, a peripheral blood sample, a pulmonary vein blood sample, a bronchopulmonary lavage sample, a whole lung lavage sample, a cerebral spinal fluid sample, or a urine sample. In some aspects, the type of liquid sample can be chosen and be relevant to the detection, prognosis and/or diagnosis of a certain type of cancer. For example, bronchopulmonary lavage or whole lung lavage can be used to detect, prognosis and/or diagnosis lung cancers); cerebral spinal fluid can be used to detect, prognosis and/or diagnosis brain metastasis (e.g., brain metastasis of lung cancers, such as small cell lung cancer; and urine can be used to detect, prognosis and/or diagnosis bladder and kidney cancers.

Biomarkers, Cell Surface Markers.

A biomarker or set of biomarkers and cell surface markers or a set of cell surface markers can be detected by the antibodies described herein, including pan-CK, EGFR, HER2, MUC1 or PLS3 and FAP and/or PD-L1. Detecting and/or identifying one or more biomarkers and/or cell surface markers can be used to indicate one or more circulating tumor cells that can be used to determine the development, presence and/or progression of cancer. Different combinations of any of the biomarkers, cell surface markers, and/or antibodies described herein can be used together in a set. The set can be used as a mixture of all or subsets of the biomarkers, cell surface markers, and/or antibodies described herein used separately in separate reactions, or immobilized in an array. Biomarkers, cell surface markers, and/or antibodies described herein used separately or as mixtures can be physically separable through, for example, association with or immobilization on a solid support. An array can include a plurality of biomarkers, cell surface markers, and/or antibodies described herein immobilized at identified or predefined locations on the array. Each predefined location on the array can generally have one type of component (that is, all the components at that location are the same). Each location can have multiple copies of the component. The spatial separation of different components in the array allows separate detection and identification of circulating tumor cells or cell surface marker-expressing cells.

Labeling.

In some aspects, any of the antibodies disclosed herein can be labeled. In some aspects, any of the antibodies disclosed herein to detect or identify one or more circulating tumor cells can be labeled. In some aspects, an anti-pan-CK antibody, an anti-EGFR antibody, an anti-HER2 antibody, an anti-MUC1 antibody and an anti-PLS3 antibody can be labeled. In some aspects, the label can be a fluorescent label. In some aspects, an anti-pan-CK antibody, an anti-EGFR antibody, an anti-HER2 antibody, an anti-MUC1 antibody and an anti-PLS3 antibody can be fluorescently labeled. In some aspects, an anti-FAP antibody and an anti-PD-L1 antibody are fluorescently labeled.

Alternatively and in some aspects, a molecular “tag” can be used, where the target probes can be attached to a detectable label, or tag, which can provide coded information about the sequence of the probe. These tags can be cleaved from the element, and subsequently detected to identify the element. In some aspects, a set of probes can be synthesized or attached to a set of coded beads, wherein each bead can be linked to a distinct probe, and wherein the beads can be coded in a manner that allows identification of the attached probe. In this type of “tag array,” flow cytometry can be used for detection of binding. For example, microspheres having fluorescence coding and can identify a particular microsphere. The probe can be covalently bound to a “color coded” object. A labeled target polypeptide can be detected by flow cytometry, and the coding on the microsphere can be used to identify the bound probe (e.g., immunoglobulin, antigen binding fragments of immunoglobulins, or ligands).

Binding.

In some aspects, the binding of any of the antibodies disclosed herein can be determined by detecting the label of the corresponding antibody. In some aspects, the binding of any of the antibodies disclosed herein to detect or identify one or more circulating tumor cells can be by detecting the label of the corresponding antibody. In some aspects, the binding of an anti-pan-CK antibody, an anti-EGFR antibody, an anti-HER2 antibody, an anti-MUC1 antibody and an anti-PLS3 antibody can be determined by detecting the label of an anti-pan-CK antibody, an anti-EGFR antibody, an anti-HER2 antibody, an anti-MUC1 antibody and an anti-PLS3 antibody. In some aspects, an detection of the label of an anti-pan-CK antibody, an anti-EGFR antibody, an anti-HER2 antibody, an anti-MUC1 antibody and an anti-PLS3 antibody can detect one or more circulating tumor cells. In some aspects, the detection of the label of ananti-FAP antibody can detect one or more circulating cancer-associated fibroblasts and/or an anti-PD-L1 antibody can detect one or more circulating tumor cells.

Contacting.

In some aspects, the contacting step can be carried out with any of the antibodies described herein. In some aspects, the contacting of the nucleated cells can be carried out with one or more of an anti-pan-CK antibody, an anti-EGFR antibody, an anti-HER2 antibody, an anti-MUC1 antibody and an anti-PLS3 antibody. In some aspects, the contacting of the nucleated cells can be carried out with any combination of an anti-pan-CK antibody, an anti-EGFR antibody, an anti-HER2 antibody, an anti-MUC1 antibody and an anti-PLS3 antibody. In some aspects, the contacting of the nucleated cells can be carried out with an anti-pan-CK antibody, an anti-EGFR antibody, an anti-HER2 antibody, an anti-MUC1 antibody and an anti-PLS3 antibody. In some aspects, the contacting of the nucleated cells with any of the antibodies disclosed herein can be conducted on a slide. In some aspects, the contacting of the nucleated cells with an anti-pan-CK antibody, an anti-EGFR antibody, an anti-HER2 antibody, an anti-MUC1 antibody and/or an anti-PLS3 antibody can be conducted on a slide. In some aspects, the contacting of the nucleated cells with an anti-FAP antibody and/or an anti-PD-L1 antibody can be conducted on a slide. In some aspects, the slide can be coated with any of the antibodies described herein. In some aspects, the slide can be coated with an anti-pan-CK antibody, an anti-EGFR antibody, an anti-HER2 antibody, an anti-MUC1 antibody and an anti-PLS3 antibody. In some aspects, the slide can be coated with an anti-cCAF antibody and/or an anti-PD-L1 antibody. In some aspects, the slide can be a glass slide, a plastic slide or a microfluidic device made of metal, glass, polydimethylsiloxane (PDMS), or plastic. In some aspects, the slide can be a microscopic slide. In some aspects, two or more of the anitbodies described herein can be on the same slide or on separate slides (e.g., two or more slides). In some aspects, an anti-pan-CK antibody, an anti-EGFR antibody, an anti-HER2 antibody, an anti-MUC1 antibody and an anti-PLS3 antibody can be on the same slide. In some aspects, increased or enhanced sensitivity of detecting circulating tumor cells can be carried out when an anti-pan-CK antibody, an anti-EGFR antibody, an anti-HER2 antibody, an anti-MUC1 antibody and an anti-PLS3 antibody are on the same slide. In some aspects, an anti-FAP antibody and an anti-PD-L1 antibody can be on the same slide or on separate slides.

Detecting.

In some aspects, the detecting of the binding of any of the antibodies disclosed herein to the nucleated cells can be conducted by scanning the slide using fiber-optic array scanning technology (FAST). In some aspects, the detecting of the binding of an anti-pan-CK antibody, an anti-EGFR antibody, an anti-HER2 antibody, an anti-MUC1 antibody and/or an anti-PLS3 antibody to the nucleated cells can be conducted by scanning the slide using FAST. In some aspects, the detecting of the binding of an anti-FAP antibody and/or an anti-PD-L1 antibody to the nucleated cells can be conducted by scanning the slide using FAST.

Disclosed herein are methods that use fiber-optic array scanning technology (FAST) to detect circulating tumor cells. Laser-printing optics are used to excite 300,000 cells/sec, and fluorescence from immuno-labels can be collected in an array of optical fibers that forms a wide collection aperture. The FAST cytometer can locate circulating tumor cells at a rate that is 500 times faster than an automated digital microscopy with comparable sensitivity and improved specificity. With this high scan rate, no enrichment of circulating tumor cells is needed. As described herein, the methods described herein can be used to measure presence and/or expression levels of a plurality of biomarkers on circulating tumor cells and/or cCAFs to prognosis and/or diagnose cancer in a subject or sample, and predict effective cancer treatment.

In some aspects, the methods disclosed herein can further comprise performing automated digital microscopy (ADM) on the cells contacted with any of the antibodies described herein. Automated digital microscopy can provide a high level of sensitivity. In some aspects, the methods disclosed herein can further comprise performing ADM on the cells contacted with an anti-pan-CK antibody, an anti-EGFR antibody, an anti-HER2 antibody, an anti-MUC1 antibody and an anti-PLS3 antibody. In some aspects, the methods disclosed herein can further comprise performing ADM on the cells contacted with an anti-FAP antibody and/or an anti-PD-L1 antibody. In some aspects, the methods disclosed herein can further comprise performing automated digital microscopy on the any of the cells positive for pan-CK, EGFR, HER2, MUC1, PLS3, FAP or PD-L1.

In some aspects, the presence or absence or expression level of one or more antibodies disclosed herein can be also be determined indirectly using RNA expression methods. Examples of RNA expression methods include but are not limited to extraction of cellular mRNA and Northern blotting using labeled probes that hybridize to transcripts encoding all or part of the gene, amplification of mRNA using gene-specific primers, polymerase chain reaction (PCR), and reverse transcriptase-polymerase chain reaction (RT-PCR), followed by quantitative detection of the gene product by a variety of methods; extraction of RNA from cells, followed by labeling, and then used to probe cDNA or oligonucleotides encoding the gene, in situ hybridization; and detection of a reporter gene.

Methods to measure protein expression levels include but are not limited to Western blot, immunoblot, ELISA, radioimmunoassay, immunoprecipitation, surface plasmon resonance, chemiluminescence, fluorescent polarization, phosphorescence, immunohistochemical analysis, microcytometry, microarray, microscopy, fluorescence activated cell sorting (FACS), and flow cytometry. The methods can also include specific protein property-based assays based including but not limited to enzymatic activity or interaction with other protein partners. Binding assays can also be used, and are well known in the art. For instance, a BIAcore machine can be used to determine the binding constant of a complex between two proteins. Other suitable assays for determining or detecting the binding of one protein to another include, immunoassays, such as ELISA and radioimmunoassays. Determining binding by monitoring the change in the spectroscopic can be used or optical properties of the proteins can be determined via fluorescence, UV absorption, circular dichroism, or nuclear magnetic resonance (NMR). Alternatively, immunoassays using specific antibody can be used to detect the expression on of a particular protein on a tumor cell.

The disclosed methods also provide for the quantification and/or qualification of susceptibility of a desired treatment based on the identification (or absence of such identification) of one or more circulating tumor cells, one or more circulating cancer-associated fibroblasts or cell surface marker-expressing cells. Said information can lead to therapeutic decisions.

Prognosis and Diagnosis.

Circulating tumor cells can be present in the blood in concentrations below 1 cell in a 100,000 white blood cells. In some aspects, circulating tumor cells can provide prognostic and diagnostic information about the underlying disease. Circulating tumor cells can comprise one or more features of metastatic tissue. Circulating cancer-associated fibroblasts can also comprise one or more features of metastatic tissue. In some aspects, the methods disclosed herein can further comprise providing a diagnosis and prognosis to a subject from which the nucleated cells were collected. In some aspects, the subject is suspected to have or is known to have cancer. In some aspects, the subject is known to have cancer and is undergoing cancer therapy. In some aspects, the cancer can be any cancer. In some aspects, the cancer can be a solid cancer or blood cancer. In some aspects, the cancer can be a lung cancer, brain metastasis of a lung cancer, bladder cancer, kidney cancer, breast cancer, melanoma, or head and neck carcinoma. Examples of lung cancers include but are not limited to small cell lung cancer. In some aspects, the methods disclosed herein can be used to determine or identify whether a subject is positive for metastatic cancerous growths or relapse of the cancer disease, post-treatment. In some aspects, the cancer can be a metastatic cancer. In some aspects, the cancer can be a recurrence of cancer, post-treatment.

It is contemplated that the sensitivity or accuracy of the methods disclosed herein can be enhanced when combining two or more of the following antibodies that can bind to two or more cell surface markers: anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody and an anti-plastin 3 (PLS3) antibody. In some aspects, the sensitivity or accuracy of the methods disclosed can further be enhanced by including anti-FAP antibody and/or an anti-PD-L1 antibody. In some aspects, the sensitivity or accuracy of the methods disclosed can further be enhanced by at least 2.5 fold compared to CK detection alone. In some aspects, the methods described herein can be useful in diagnosing the presence of cancer (e.g., a lung cancer) at different stages of the disease as well as determining the responsiveness of the subject to a cancer therapy. The presence of circulating metastatic tumor stem cells in a sample obtained from the subject can be identified by measuring the presence of one or more pan-CK, EGFR, HER2, MUC1 and PLS3 positive circulating tumor stem cells, which can be diagnostic/prognostic for the presence of metastatic cancerous tumors.

Responsiveness to Cancer Therapy.

In some aspects, the methods disclosed herein can further comprise determining the responsiveness of a subject to a cancer therapy. In some aspects, the cancer therapy can be a PD-L1 inhibitor. In some aspects, the detection of an anti-PD-L1 antibody in a sample or in the nucleated cells obtained from a subject or the presence of an anti-PD-L1 antibody in a sample or in the nucleated cells obtained from a subject can indicate that the subject will respond to be PD-L1 inhibitor. In some aspects, the PD-L1-inhibitor can be atezolizumab, durvalumab, or avelumab.

### Compositions

Disclosed herein are compositions that can be useful in detecting one or more circulating tumor cells. In some aspects, the compositions can also be useful in detecting one or more circulating cancer-associated fibroblasts. Also, disclosed herein are compositions that can be useful in determining the responsiveness of a subject to a cancer therapy. Further disclosed herein are compositions that can be useful diagnosing and prognosing cancer or metastasis in a subject. In some aspects, the compositions disclosed herein can be useful in diagnosing non-small cell lung cancer in a subject. Also, disclosed herein are compositions that can be useful in detecting and treating non-small-cell lung cancer in a subject.

Disclosed herein are compositions comprising two or more antibodies selected from the group consisting of an anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody, an anti-plastin 3 (PLS3) antibody, an anti-FAP antibody, and an anti-PD-L1 antibody. In some aspects, the two or more of the compositions disclosed herein can be included as part of a comprehensive tumor cell detection marker panel (or array) or a tumor cell detection marker panel (or array). In some aspects, the tumor cell detection marker panel or arrays can be two or more antibodies selected from the group consisting of an anti-pan-CK antibody, an anti-EGFR antibody, an anti-HER2 antibody, an anti-MUC1 antibody, an anti-PLS3 antibody, an anti-cCAF antibody, and an anti-PD-L1 antibody. In some aspects, the tumor cell detection marker panel or array can be any combination of two or more antibodies selected from the group consisting of an anti-pan-CK antibody, an anti-EGFR antibody, an anti-HER2 antibody, an anti-MUC1 antibody, and an anti-PLS3 antibody. In some aspects, one or more of the antibodies can be labeled. In some aspects the antibodies can be the pan-CK antibody (Sigma, C2562), CK-19 antibody (Dako, M0888), MUC1 antibody (Biolegend, 355602), EGFR antibody (Santa Cruz, sc365829), HER2 antibody (Biolegend, 324402), or the Plastin3 antibody (Abnova, H00005358-M01).

The tumor cell detection marker panel or array disclosed herein comprising two or more antibodies selected from the group consisting of an anti-pan-CK antibody, an anti-EGFR antibody, an anti-HER2 antibody, an anti-MUC1 antibody, and an anti-PLS3 antibody can increase the sensitivity of any of the methods disclosed herein. The tumor cell detection marker panel or array disclosed herein comprising at least an anti-pan-CK antibody, an anti-EGFR antibody, an anti-HER2 antibody, an anti-MUC1 antibody, an anti-PLS3 antibody can increase the sensitivity of any of the methods disclosed herein to at least 88.5%. In some aspects, the increased or higher sensitivity can be without pre-enrichment. In some aspects, the compositions and methods disclosed herein can provide information on epithelial circulating tumor cells and/or mesenchymal circulating tumor cells and/or circulating cancer-associated fibroblasts.

The tumor cell detection marker panel or array disclosed herein can be used in a standalone method for detecting one or more circulating tumor cells, detecting one or more circulating cancer-associated fibroblasts, determining the responsiveness of a subject to a cancer therapy, diagnosing and prognosing cancer or metastasis diagnosing non-small cell lung cancer, and/or detecting and treating non-small-cell lung cancer in a sample and/or subject or in combination with one or more other panels (e.g., gene protein expression panels or arrays or with one or more biomarkers or cell surface markers) not disclosed herein. The tumor cell detection marker panel or array disclosed herein can be used along with one or more diagnostic tests.

In some aspects, the tumor cell detection marker panel or array disclosed herein comprising two or more antibodies selected from the group consisting of an anti-pan-CK antibody, an anti-EGFR antibody, an anti-HER2 antibody, an anti-MUC1 antibody, an anti-PLS3 antibody, an anti-FAP antibody, and an anti-PD-L1 antibody can further comprise one or more additional antibodies or tumor cell detection markers. In some aspects, the one or more additional antibodies or tumor cell detection markers can be an anti-FAP antibody and/or an anti-PD-L1 antibody. The tumor cell detection marker panel or array disclosed herein can also be used in methods to generate a specific profile. In some aspects, the profile generated can be used to determine the status of a cancer in the subject. In some aspects, the tumor cell detection marker panel or array disclosed herein can also be used to compare two or more samples. In some aspects, the second sample can be obtained at any time point after the first sample is obtained. In some aspects, the increase in the number of tumor cells detected that express two or more of the antibodies in the tumor cell detection marker panel or array can indicate that a cancer is progressing in the subject. In some aspects, a decrease in the number of tumor cells detected that express the two or more of the antibodies in the tumor cell detection marker panel or array can indicate that cancer is regressing in the subject. In some aspects, no significant change in the number of tumor cells that express two or more of the antibodies in the tumor cell detection marker panel or array can indicate that cancer is stable in the subject. In some aspects, the quantity or amount of the number of tumor cells that express two or more of the antibodies in the tumor cell detection marker panel or array can be used to compute a statistically significant value based on differential expression of the two or more antibodies in the tumor cell detection marker panel or array disclosed herein, wherein the computed value correlates to a diagnosis for a status of the cancer. The variance in the obtained profile of the expression levels or amount of the selected antibodies in the tumor cell detection marker panel or array can be either increased or decreased compared to an initial or first sample obtained from the subject or compared to reference subject or control.

In some aspects, the profile generated using the tumor cell detection marker panel or array can be used for cancer immunotherapy companion diagnostics. In some aspects, the tumor cell detection marker panel or array disclosed herein can be further include an anti-PD-L1 antibody to determine or assess the responsiveness of a subject to a cancer therapy. In some aspects, the number of CTCs, the expression level of PD-L1 on the CTCs and/or the percentage of CTCs positive for PD-L1 expression can be computed, evaluated and/or used to determine a subject's response to a cancer therapy. In some aspects, the presence of PD-L1 on one or more CTCs indicates that a subject will respond to a cancer immunotherapy. In some aspects, the higher the number of CTCs or percentage of CTCs positive for PD-L1 indicate that a subject will respond to a cancer therapy. In some aspects, the cancer therapy can be a PD-L1 inhibitor. In some aspects, the PD-L1-inhibitor can be atezolizumab, durvalumab, or avelumab.

Different compositions can be used together as a set. The set can be used as a mixture of all or subsets of the compositions used separately in separate reactions, or immobilized in an array. Compositions used separately or as mixtures can be physically separable through, for example, association with or immobilization on a solid support. An array can include a plurality of compositions immobilized at identified or predefined locations on the array. Each predefined location on the array can generally have one type of component (that is, all the components at that location are the same). Each location can have multiple copies of the component. The spatial separation of different components in the array allows separate detection and identification of the antibodies or polypeptides disclosed herein.

One of ordinary skill in the art can determine the amount or expression level of one or more biomarkers (or proteins) disclosed herein any number of ways. To detect or quantify the level of RNA products of the biomarkers within a sample, arrays, such as microarrays, RT-PCR (including quantitative RT-PCR), nuclease protection assays and Northern blot analyses can be used. Accordingly, in some aspects, the biomarker expression levels can be determined using arrays, microarrays, RT-PCR, quantitative RT-PCR, nuclease protection assays or Northern blot analyses.

An array is a form of solid support. An array detector is also a form of solid support to which multiple different capture compositions or compounds or detection compositions or compounds have been coupled in an array, grid, or other organized pattern.

Solid-state substrates for use in solid supports can include, for instance, any solid material to which molecules can be coupled. Examples of such materials include acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, poly lactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or any combination thereof. Solid-state substrates and solid supports can be porous or non-porous. An example of a solid-state substrate is a microtiter dish (e.g., a standard 96-well type). A multiwell glass slide can also be used. For example, such as one containing one array per well can be used, allowing for greater control of assay reproducibility, increased throughput and sample handling, and ease of automation.

It is not required that a given array be a single unit or structure. The set of compositions can be distributed over any number of solid supports. For example, each composition can be immobilized in a separate reaction tube or container, on separate beads or microparticles, or on separate slides. Different aspects of the disclosed method and use of the tumor cell detection marker panel or array can be performed with different components (e.g., different compositions specific for different proteins) immobilized on a solid support.

Methods for immobilizing nucleic acids, peptides or antibodies (and other proteins) to solid-state substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidinbiotin, photocrosslinkable agents, epoxides, maleimides and N-[y-Maleimidobutyryloxy] succinimide ester (GMBS), and a heterobifunctional crosslinker. Antibodies can be attached to a substrate by chemically cross-linking a free amino group on the antibody to reactive side groups present within the solid-state substrate. Antibodies can be, for example, chemically cross-linked to a substrate that contains free amino, carboxyl, or sulfur groups using glutaraldehyde, carbodiimides, or GMBS, respectively, as cross-linker agents. In this method, aqueous solutions containing free antibodies can be incubated with the solid-state substrate in the presence of glutaraldehyde or carbodiimide.

A method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS3) or a heterobifunctional cross-linker agent such as GMBS. For crosslinking with GMBS, glass substrates can be chemically functionalized by immersing in a solution of mercaptopropyltrimethoxysilane (1% vol/vol in 95% ethanol pH 5.5) for 1 hour, rinsing in 95% ethanol and heating at 120° C. for 4 hrs. Thiol-derivatized slides can be activated by immersing in a 0.5 mg/mL solution of GMBS in 1% dimethylformamide, 99% ethanol for 1 hour at room temperature. Antibodies or proteins can be added directly to the activated substrate, which can be blocked with solutions containing agents such as 2% bovine serum albumin, and air-dried. Other standard immobilization chemistries are known by those of ordinary skill in the art.

Each of the components (e.g., compositions) immobilized on the solid support can be located in a different predefined region of the solid support. Each of the different predefined regions can be physically separated from each other. The distance between the different predefined regions of the solid support can be either fixed or variable. For example, in an array, each of the compositions can be arranged at fixed distances from each other, while components associated with beads will not be in a fixed spatial relationship. The use of multiple solid support units (e.g., multiple beads) can result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

Disclosed herein, are solid supports comprising one or more primers, probes, polypeptides, or antibodies capable of hybridizing or binding to one or more of the genes disclosed herein. Solid supports are solid state substrates or supports that molecules, such as analytes and analyte binding molecules, can be associated. Analytes (e.g., calcifying nano-particles and proteins) can be associated with solid supports directly or indirectly. For example, analytes can be directly immobilized on solid supports. Analyte capture agents (e.g., antibodies, capture compounds) can also be immobilized on solid supports.

In some aspects, the methods described herein can be performed over time, and the change in the level of one or more of the biomarkers or compositions can be assessed. For example, the assays can be performed every 24-72 hours for a period of 6 months to 1 year, and thereafter carried out as needed. Assays can also be completed prior to, during, or after a treatment protocol. Together, the tumor cell detection marker panel or array disclosed herein can be used to profile an individual's risk or progression of a cancer. As used within this context, the terms “differentially expressed” or “differential expression refers to difference in the level of expression or amount of the one or more of biomarkers or compositions of the tumor cell detection marker panel or array disclosed herein that can be assayed by measuring the level of expression of the products (e.g., RNA) of the one or more of biomarkers or compositions of the tumor cell detection marker panel or array, such as the difference in level of messenger RNA transcript or a portion thereof expressed or of proteins expressed of the one or more of biomarkers or compositions of the tumor cell detection marker panel or array. In some aspects, this difference is significantly different.

Levels of expression can be measured at the transcriptional and/or translational levels. At the translational level, expression of any of the biomarkers or compositions of the tumor cell detection marker panel or array described herein can be measured using immunoassays including immunohistochemical staining, western blotting, ELISA and the like with an antibody that selectively binds to the corresponding protein. Detection of the protein using protein-specific antibodies in immunoassays is known in the art. At the transcriptional level, mRNA can be detected by, for example, amplification (e.g., PCR, LCR), or hybridization assays (e.g., northern hybridization, RNAse protection, or dot blotting). The level of protein or mRNA can be detected, for example, by using directly or indirectly labeled detection agents (e.g., fluorescently or radioactively labeled nucleic acids, radioactively or enzymatically labeled antibodies).

### Kits

In some aspects, a kit is disclosed comprising two or more antibodies selected from the group consisting of an anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody, and an anti-plastin 3 (PLS3) antibody. In some aspects, a kit is disclosed comprising an anti-FAP antibody and/or an anti-PD-L1 antibody.

In some aspects, the tumor cell detection marker panel or array can consist of primers or probes capable of detecting, amplifying or otherwise measuring the presence or expression of one or more of the biomarkers (pan-CK, EGFR, HER2, MUC1, PLS3, FAP and/or PD-L1). In some aspects, a diagnostics kit is disclosed comprising one or more probes or primers capable of detecting, amplifying or measuring the presence or expression of one or more pan-CK, EGFR, HER2, MUC1, PLS3, FAP and/or PD-L1 disclosed herein.

In some aspects, the kits described herein can also comprise one or more PD-L1 inhibitors. In some aspects, the PD-L1-inhibitor can be atezolizumab, durvalumab, or avelumab.

The kits described herein can include any combination of the compositions (e.g., antibodies for pan-CK, EGFR, HER2, MUC1, PLS3, FAP and PD-L1) described above and suitable instructions (e.g., written and/or provided as audio-, visual-, or audiovisual material). In some aspects, the kit comprises a predetermined amount of a composition comprising any one of the antibodies for pan-CK, EGFR, HER2, MUC1, PLS3, FAP and PD-L1 and/or PD-L1 inhibitors disclosed herein. The kit can further comprise one or more of the following: instructions, sterile fluid, syringes, a sterile container, delivery devices, slides, solid supports, and buffers or other control reagents.

### EXAMPLES

### Example 1: Development of a Highly Sensitive Liquid Biopsy Test for Non-Small Cell Lung Cancer (NSCLC) Immunotherapy

A fiberoptic array scanning technology (FAST) to detect CTC based on fluorescence staining has been developed (Somlo, G., et al., Breast cancer research and treatment, 2011. 128(1): p. 155-163; and Ao, Z. and X. Liu, Fiber-optic array scanning technology (fast) for detection and molecular characterization of circulating tumor cells, in Circulating Tumor Cells. 2017, Springer. p. 235-246). Benefiting from its laser scanning array design, the fluorescence staining could be interrogated at an ultra-fast speed of 20 million cells per minute. Then the location of target cells could be identified on the slide followed by performing a detailed automated digital microscopy (ADM) on single cells at high resolution. This technology is 500 times faster than conventional ADM (Krivacic, R. T., et al., Proc Natl Acad Sci USA, 2004. 101(29): p. 10501-4). This technology has provided an uncommon advantage to enumerate CTC from vast number (˜100 million) of peripheral blood mononuclear cells (PBMC) from a standard 7.5 mL blood draw without the requirement of any format of pre-enrichment either biased by protein marker expression or size exclusion.

To increase the sensitivity of CTC tests using FAST technology, a comprehensive tumor cell detection marker panel was developed including pan-cytokeratin (pan-CK), epidermal growth factor receptor (EGFR), human epidermal growth factor receptor 2 (HER2), mucin 1 (MUC1) and plastin 3 (PLS3). Using this panel, mesenchymal CTCs were successfully detected. Circulating cancer associated fibroblasts (cCAF) were also incorporated as an additional liquid biopsy marker for NSCLC. Circulating CAF has been reported in breast cancer and colorectal cancer and shown to be disease specific marker (Ao, Z., et al., Cancer Res, 2015. 75(22): p. 4681-7). cCAF can be detected by FAST using FAP as marker. Combined, the overall sensitivity of the disclosed liquid biopsy test using the comprehensive tumor cell detection marker panel was increased to 88.5%. Finally, Programmed death-ligand 1 (PD-L1) expression level on CTC was characterized using a clinically validated anti-PD-L1 antibody (Ventana SP-263), proving the utility of the disclosed tests in serving as companion diagnostics for ICI treatment.

Materials and Methods.

Patient Blood Collection and Sample Preparation.

Samples from patients with advanced stage non-small cell lung cancer (NSCLC) who consented to participate in the research were collected. Two tubes of blood from each patient were collected in Cell-Free DNA BCT tubes (Streck, USA). Collected blood was then processed within 12 hours. Firstly, red blood cells from blood samples were lysed with ACK lysis buffer for 5 minutes (Invitrogen, USA). Nucleated cells from blood were then pelleted and counted on a hematocytometer. After counting, cells were then plated on a section of large (10.8 cm×7.6 cm) custom-designed adhesive slides (Marienfeld, Bad Mergentheim, Germany) for 40 minutes to allow cells to adhere. Adhered cells were then fixed with 2% PFA and cold acetone. Acetone fixed slides can be immediately stained or stored at −80° C. for up to 6 months before analysis.

Immunofluorescence Staining of CTC and cCAF.

For CTC detection, the adhered cells were first incubated with primary antibody at following concentrations: pan-CK (Sigma, C2562) 1:100, CK-19 (Dako, M0888) 1:100, MUC1 (Biolegend, 355602) 1:200, EGFR (Santa Cruz, sc365829, 1:200), HER2 (Biolegend, 324402) 1:200, Plastin3 (Abnova, H00005358-M01) 1:50, Tumor markers were then labeled with Biotin-anti-IgG1 (Life Technologies, A10519) followed with streptavidin-Alexa 555 (Life Technologies, S32355). Leukocytes were labeled with pre-conjugated CD45-Q800 (Thermo, customized) at 1:50. For secondary markers, CTCs were labeled with anti-vimentin (AbCAM, EPR3776) at 1:800 or anti-PD-L1 (Ventana, SP263, pre-diluted), followed by secondary antibody staining anti-rabbit-Alexa647 (Life Technologies, A21244) at 1:400. For cCAF detection, adhered cells were incubated with primary antibody anti-FAP (R&D Biosystems, MAB3715) 1:200 and anti-alpha-smooth-muscle-actin (AbCAM, AB5694) 1:400 followed by secondary and tertiary antibody staining. Samples were then counterstained with DAPI (Life Technologies) and coverslipped with mounting media (Vector Laboratories).

FAST Detection of CTC and cCAF and Automated Digital Microscopy (ADM).

FAST detection was performed as described in Ao, Z. and X. Liu, Fiber-optic array scanning technology (fast) for detection and molecular characterization of circulating tumor cells, in Circulating Tumor Cells. 2017, Springer. p. 235-246. Briefly, immunofluorescently stained slides were placed on FAST scanner with fiduciary markers to calibrate coordinate. Slides were scanned on FAST scanner for <1 minute for automated target cell position detection as “hits”. Detected target hit list and coordinates were then transferred to an automated microscope. Detected hit positions will then get imaged at 200× magnification with automated digital microscopy (ADM) in four channels: DAPI, FITC, TRITC and Qdot800.

Cell Culture and Passaging.

Non-small cell lung cancer (NSCLC) cell lines for immunofluorescence staining were purchased from American Type Culture Collection (ATCC) including A549, H2228, H522, H820, H441, H23 cells. Cells were cultured in RPMI-1640 medium supplemented with 10% fetal bovine serum (FBS) and maintained in humidified incubator with 5% CO2 environment. CAF-21 cells were maintained in IMEM medium (Gibco, USA) supplemented with 10% fetal bovine serum (FBS).

Results.

Development of Multiplexed Assay for Mesenchymal CTC Detection.

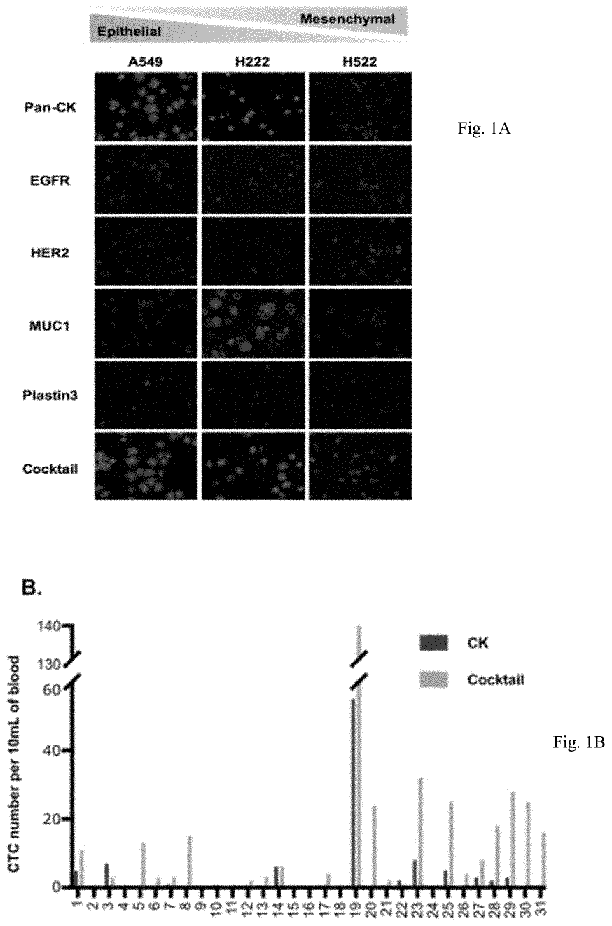

To develop a multiplexed assay detecting mesenchymal CTC, three human NSCLC cell lines were adopted with gradual epithelial to mesenchymal status spectrum, namely A549 (epithelial), H2228 (moderate epithelial) and H522 (mesenchymal). As shown in FIG. 1A, H522 does not have detectable cytokeratin expression, which would be left out by any cytokeratin-based CTC identification method. To effectively detect all tumor cells, individual marker expression of pan-CK. EGFR, HER2, MUC1 and PLS3 were interrogated on these 3 tumor cell lines. As shown in FIG. 1, A549 has high pan-CK expression and moderate EGFR and PLS3 expression, H2228 has moderate pan-CK, EGFR expression and high MUC1 expression, whereas H522 has moderate EGFR and HER2 expression. When combined together as a tumor cell detection marker panel comprising pan-cytokeratin (pan-CK), epidermal growth factor receptor (EGFR), human epidermal growth factor receptor 2 (HER2), mucin 1 (MUC1) and plastin 3 (PLS3), the multiplexed assay can effectively detect the 3 cell lines effectively (FIG. 1A).

Validation of Multiplexed Assay for Mesenchymal CTC Detection in Clinical Cases.

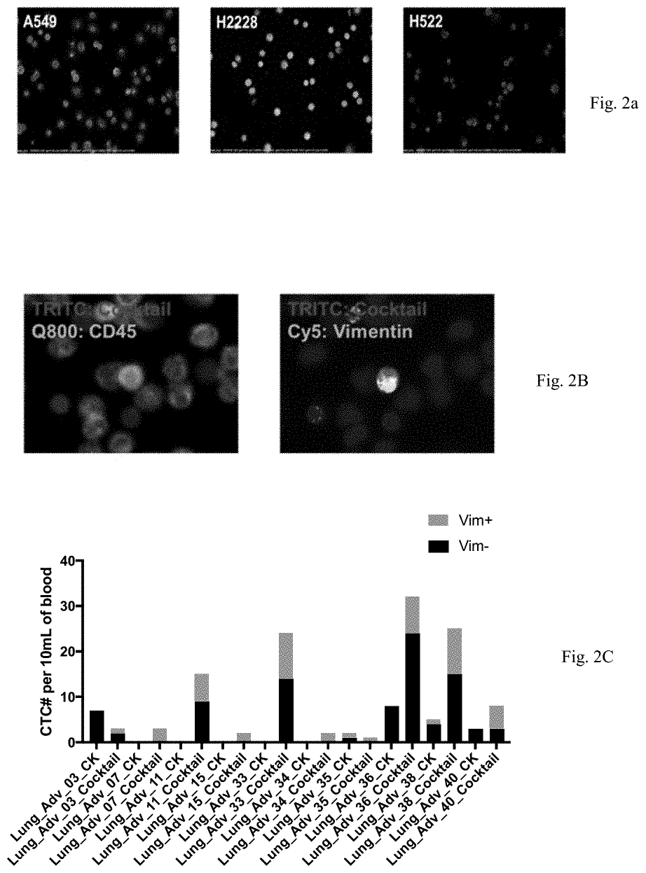

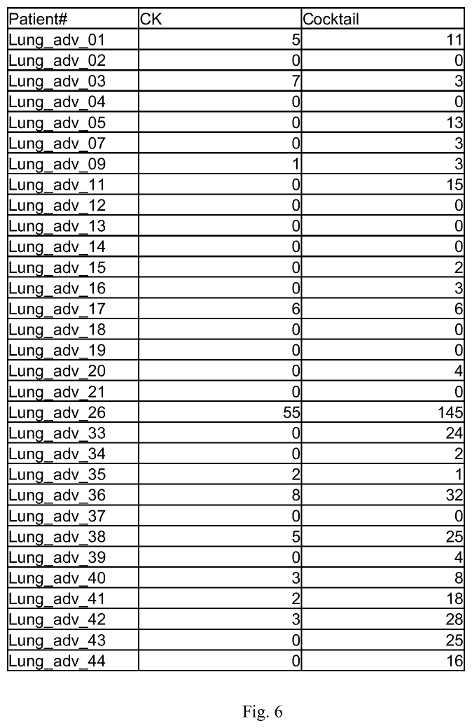

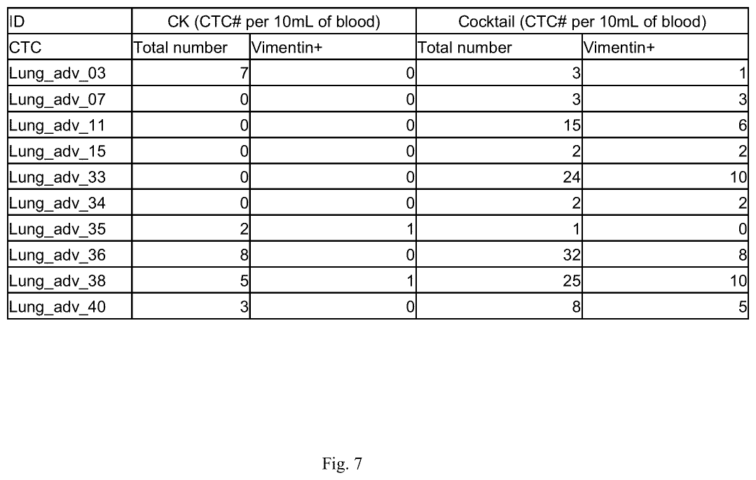

Because the disclosed tumor cell detection marker panel of antibodies, comprising an anti-pan-CK antibody, an anti-EGFR antibody, an anti-HER2 antibody, an anti-MUC1 antibody and an anti-PLS3 antibody, showed superior detection sensitivity especially for mesenchymal CTC on cell line studies, the disclosed tumor cell detection marker panel of antibodies, comprising an anti-pan-CK antibody, an anti-EGFR antibody, an anti-HER2 antibody, an anti-MUC1 antibody and an anti-PLS3 antibody, was tested on clinical samples. This tumor cell detection marker panel of antibodies, comprising an anti-pan-CK antibody, an anti-EGFR antibody, an anti-HER2 antibody, an anti-MUC1 antibody and an anti-PLS3 antibody, was applied on a pilot cohort of 31 NSCLC patients. CTC was detected in 35.5% of the patients by traditional cytokeratin staining and 71.0% by the disclosed tumor cell detection marker panel of antibodies comprising an anti-pan-CK antibody, an anti-EGFR antibody, an anti-HER2 antibody, an anti-MUC1 antibody and an anti-PLS3 antibody (FIG. 1B, FIG. 6). This demonstrated the superior sensitivity of the disclosed assay. In addition, the EMT status of the CTCs detected by the disclosed tumor cell detection marker panel of antibodies, comprising an anti-pan-CK antibody, an anti-EGFR antibody, an anti-HER2 antibody, an anti-MUC1 antibody and an anti-PLS3 antibody, was examined, showing that 41.7% of the CTCs detected by the disclosed tumor cell detection marker panel of antibodies, comprising an anti-pan-CK antibody, an anti-EGFR antibody, an anti-HER2 antibody, an anti-MUC1 antibody and an anti-PLS3 antibody, were Vimentin+, whereas in CK detection samples, the vimentin+CTC was 8%. (FIG. 2, FIG. 7). Moreover, of the samples where CK antibody alone failed to detect CTCs but the tumor cell detection marker panel of antibodies, comprising an anti-pan-CK antibody, an anti-EGFR antibody, an anti-HER2 antibody, an anti-MUC1 antibody and an anti-PLS3 antibody, detected CTCs, the tumor cell detection marker panel of antibodies, comprising an anti-pan-CK antibody, an anti-EGFR antibody, an anti-HER2 antibody, an anti-MUC1 antibody and an anti-PLS3 antibody, detected CTCs contained mesenchymal vimentin+CTC. This finding demonstrated that the disclosed tumor cell detection marker panel of antibodies, comprising an anti-pan-CK antibody, an anti-EGFR antibody, an anti-HER2 antibody, an anti-MUC1 antibody and an anti-PLS3 antibody, enhanced the sensitivity of NSCLC CTC detection by detecting mesenchymal CTC that was “invisible” to CK alone detection methods.

Detection of cCAF Increase Sensitivity of Liquid Biopsy Test.

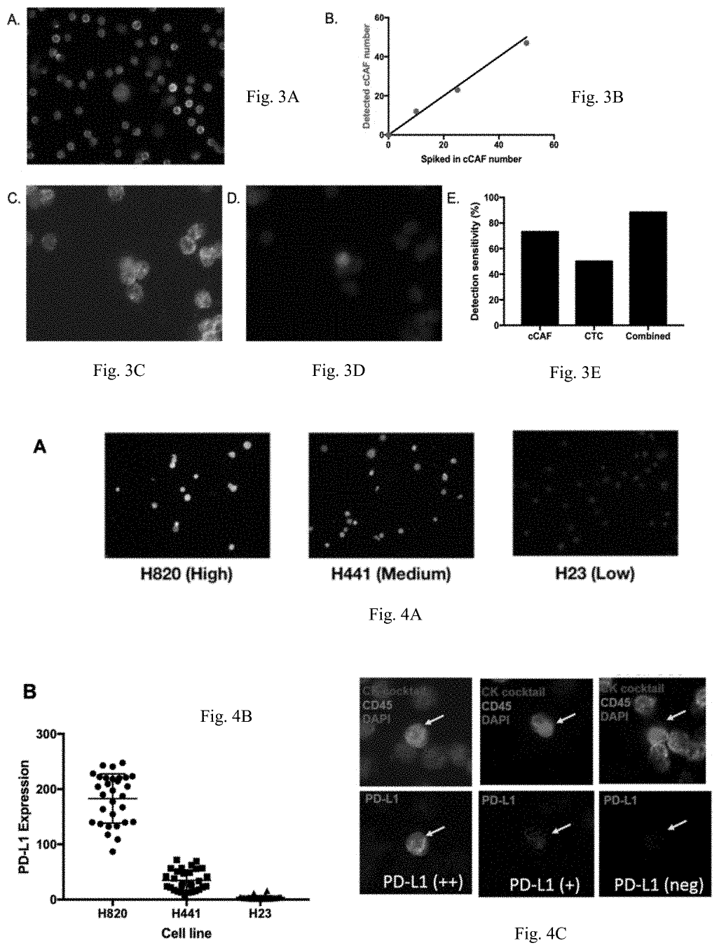

Previously, circulating cancer associated fibroblasts (cCAF) were shown to be detected in blood samples collected from breast, colorectal and prostate cancer patients. Especially in breast cancer, cCAF can be detected in early stage (Ao, Z., et al., Cancer Res, 2015. 75(22): p. 4681-7). To test whether cCAF can be detected in NSCLC patients, a FAST-based test was developed for cCAF. Briefly, CAF21 cells derived from breast cancer patient primary tumor (Drews-Elger, K., et al., Breast Cancer Res Treat, 2014. 144(3): p. 503-17) were spiked in PBMC isolated from healthy donors and stained for fibroblast activation protein (FAP) as primary detection antibody (FIG. 3A). CAF was identified as FAP+CK-nucleated cells with intact morphology. FAST detection sensitivity of cCAF was validated at 10 cells per 10 mL of blood (FIG. 3B). cCAF level was interrogated in 26 advanced stage NSCLC patients. cCAF was detected in NSCLC blood sample as FAP+, CK−, CD45-nucleated cells (FIG. 3 C,D). cCAF was detected 13 (50.0%) of metastatic NSCLC patients. Then CTC detection sensitivity was compared to the disclosed tumor cell detection marker panel of antibodies, comprising an anti-pan-CK antibody, an anti-EGFR antibody, an anti-HER2 antibody, an anti-MUC1 antibody and an anti-PLS3 antibody, cCAF detection sensitivity as well as CTC/cCAF combined. CTC was detected in 19/26 (73.1%) of metastatic NSCLC patients, cCAF was detected in 13/26 (50%) of patients. Then CTC/cCAF was combined together as liquid biopsy, to detect NSCLC with at least 1 CTC or 1 cCAF at a sensitivity of 23/26 (88.4%) (FIG. 3E).

Analysis of PD-L1 Expression Level on CTC of NSCLC Patients.

Cancer immunotherapy, especially immune-checkpoint inhibitors (ICI) such as anti-PD-1 antibody has achieved tremendous success in epithelial cancers such as NSCLC and melanoma. CTC holds great potential as companion diagnostics for anti-PD1/PD-L1 therapy since it reflects PD-L1 expression status of the tumor cells in real time and can be re-sampled multiple times before and during ICI treatment, and may represent better the metastatic site (Janning, M., et al., Cancers, 2019. 11(6): p. 835). Thus, it is of great interest to develop CTC based companion diagnostics for ICI.

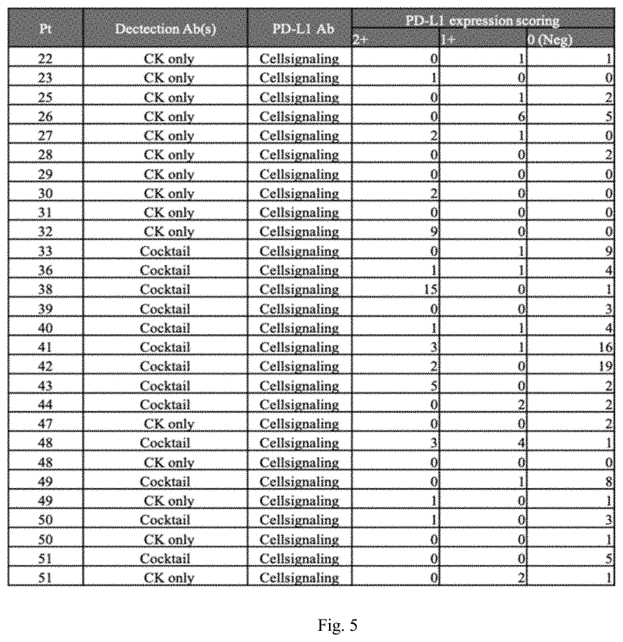

To develop a FAST-based CTC PD-L1 expression level test, the disclosed cocktail, comprising an anti-pan-CK antibody, an anti-EGFR antibody, an anti-HER2 antibody, an anti-MUC1 antibody and an anti-PLS3 antibody, was used to detect CTC in NSCLC patients. The detected CTCs were then analyzed for PD-L1 expression using a FDA-approved cDx antibody Ventana SP263. This antibody was validated using 3 NSCLC cell lines with low, medium and high PD-L1 expression. As shown in FIG. 5A, Ventana SP263 can distinguish PD-L1 expression levels in these 3 cell lines using a quantitative method (FIG. 5V). After this antibody was validated, it was applied to 28 samples collected from 24 of NSCLC patients. PD-L1 expression was quantified on these patient CTCs. As shown in FIG. 5, within the same patient, CTC PD-L1 expression level can be highly heterogenous.

Discussion

Sensitive detection of CTC in NSCLC has been a challenge due to the high EMT status of NSCLC CTCs (Milano, A., et al., Anal Cell Pathol (Amst), 2018. 2018: p. 3506874). FDA-cleared Cellsearch technology can detect CTC in 27% of the advanced stage NSCLC patients (Hofman, V., et al., Clin Cancer Res, 2011. 17(4): p. 827-35). Disclosed herein is a FAST based assay to detect CTC at sensitivity of, for example, 71.0% in metastatic NSCLC patients' blood. The disclosed methods combine the strength of ultra-high throughput detection of CTC using the FAST system, with ultra-sensitive detection capacity of the disclosed cancer marker panel. The detection sensitivity is superior as compared with other reports using antigen based or size based enrichment (Shishido, S. N., et al., Journal of Translational Medicine, 2019. 17(1): p. 294; and Krebs, M. G., et al., J Clin Oncol, 2011. 29(12): p. 1556-63). Moreover, although circulating fibroblasts progenitor cells have been reported before in lung cancer setting (Ishii, G., et al., STEM CELLS, 2007. 25(6): p. 1469-1477). Thorough investigation on these cells with systemic detection methodology have yet been developed. These compositions and methods demonstrate that using FAST's no-pre-enrichment method, circulating cancer associated fibroblasts (cCAF) in lung cancer setting was detected with high sensitivity (as few as 1 cell per 1 milliliter of blood). The disclosed compositions and methods also demonstrated that cCAF can be detected in 50% of NSCLC patients. In addition, when combined with the disclosed ultrasensitive CTC detection technology, the liquid biopsy sensitivity to detect advanced stage NSCLC can reach 88.4%. The disclosed tumor cell detection marker panel of antibodies, comprising an anti-pan-CK antibody, an anti-EGFR antibody, an anti-HER2 antibody, an anti-MUC1 antibody and an anti-PLS3 antibody, can also be used on early stage lung cancer patients.

In addition to the ultra-high sensitivity of NSCLC detection by liquid biopsy, a FDA-approved cDx antibody Ventana SP263 was used to quantify PD-L1 expression on CTC. Previous reports by antigen based or sized based enrichment of CTC have shown that PD-L1 expression on CTC could predict responses to Nivolumab (anti-PD-1) (Adams, D. L., et al., Clin Cancer Res, 2017. 23(19): p. 5948-5958; Janning, M., et al., Cancers, 2019. 11(6): p. 835; Nicolazzo, C., et al., Scientific reports, 2016. 6: p. 31726; Kallergi, G., et al., Therapeutic advances in medical oncology, 2018. 10: p. 1758834017750121; Wang, Y., et al., Scientific reports, 2019. 9(1): p. 566; Ilié, M., et al., Annals of Oncology, 2017. 29(1): p. 193-199; and Dhar, M., et al., Scientific reports, 2018. 8(1): p. 2592). Here, the data demonstrated that PD-L1 expression can be analyzed on NSCLC CTC using the FAST platform. Utilizing the FAST platform, CTC can be detected with higher sensitivity without pre-enrichment, thus this technology could provide more information on both epithelial and mesenchymal CTCs' PD-L1 expression status. In concordance with others' report, heterogeneous expression of PD-L1 on CTC was detected. To conclude, the disclosed FAST-based method provides an ultrasensitive method to detect NSCLC and can be used for cancer immunotherapy companion diagnostics.

1. A method of detecting one or more circulating tumor cells, the method comprising:
obtaining or having obtained nucleated cells from a subject;
contacting the nucleated cells with an anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody and an anti-plastin 3 (PLS3) antibody; and
detecting binding of the anti-pan-CK antibody, the anti-EGFR antibody, the anti-HER2 antibody, the anti-MUC1 antibody and the anti-PLS3 antibody to the nucleated cells, wherein binding of the anti-pan-CK antibody, the anti-EGFR antibody, the anti-HER2 antibody, the anti-MUC1 antibody and the anti-PLS3 antibody to the nucleated cells detects one or more circulating tumor cells.

2. The method of claim 1, wherein the anti-pan-CK antibody, the anti-EGFR antibody, the anti-HER2 antibody, the anti-MUC1 antibody and the anti-PLS3 antibody are labeled.

3. The method of claim 2, wherein the binding of the anti-pan-CK antibody, the anti-EGFR antibody, the anti-HER2 antibody, the anti-MUC1 antibody and the anti-PLS3 antibody is determined by detecting the label of the anti-pan-CK antibody, the anti-EGFR antibody, the anti-HER2 antibody, the anti-MUC1 antibody and the anti-PLS3 antibody.

4. The method of claim 3, wherein the detection of the label of the anti-pan-CK antibody, the anti-EGFR antibody, the anti-HER2 antibody, the anti-MUC1 antibody and the anti-PLS3 antibody detects one or more circulating tumor cells.

5. The method of claim 1, further comprising contacting the nucleated cells with an anti-FAP antibody to detect one or more circulating cancer-associated fibroblasts (cCAFs).

6. The method of claim 5, further comprising detecting the FAP antibody.

7. The method of claim 1, further comprising contacting the nucleated cells with an anti-programmed death-ligand (PD-L1) antibody.

8. The method of claim 7, further comprising detecting the anti-PD-L1 antibody.

9. (canceled)

10. (canceled)

11. The method of claim 1, wherein the contacting of the nucleated cells with the anti-pan-CK antibody, the anti-EGFR antibody, the anti-HER2 antibody, the anti-MUC1 antibody and the anti-PLS3 antibody is conducted on a slide.

12. (canceled)

13. The method of claim 1, wherein the detecting of the binding of the anti-pan-CK antibody, the anti-EGFR antibody, the anti-HER2 antibody, the anti-MUC1 antibody and the anti-PLS3 antibody to the nucleated cells is conducted by scanning the slide using fiber-optic array scanning technology (FAST).

14. The method of claim 13, further comprising performing automated digital microscopy (ADM) on the cells contacted with the anti-pan-CK antibody, the anti-EGFR antibody, the anti-HER2 antibody, the anti-MUC1 antibody and the anti-PLS3 antibody.

15. (canceled)

16. (canceled)

17. (canceled)

18. The method of claim 1, wherein the subject is suspected to have or is known to have cancer.

19. (canceled)

20. The method of claim 1, further comprising determining the responsiveness of the subject to a cancer therapy, wherein the cancer therapy is a PD-L1 inhibitor.

21. (canceled)

22. (canceled)

23. The method of claim 1, wherein the circulating tumor cell is a non-small-cell lung cancer cell.

24. (canceled)

25. (canceled)

26. (canceled)

27. A method of diagnosing non-small cell lung cancer in a subject, the method comprising:
obtaining or having obtained nucleated cells from a subject;
contacting the nucleated cells with a slide comprising a surface coated with antibodies that bind one or more cell surface markers pan-cytokeratin (pan-CK), epidermal growth factor receptor (EGFR), human epidermal growth factor receptor 2 (HER2), mucin 1 (MUC1) and plastin 3 (PLS3);
detecting the presence of immunostaining of the nucleated cells with the antibodies by scanning the slide using fiber-optic array scanning technology (FAST), wherein a cell positive for pan-CK, EGFR, HER2, MUC1 or PLS3 is a circulating tumor cell; and
identifying the subject as having non-small-cell lung cancer based on the presence of the circulating tumor cell that expresses the one or more cell surface markers in the sample.

28. The method of claim 27, further comprising providing a subsequent sample comprising blood from the subject; separating nucleated cells from the blood; and quantifying a level of cell surface marker-expressing cells in the first and subsequent samples, wherein an increase in the number of cells that express the cell surface markers indicates that cancer is progressing in the subject; a decrease in the number of cells that express the surface markers indicates that cancer is regressing in the subject; and no significant change in the number of cells that express the surface markers indicates that cancer is stable in the subject.

29. (canceled)

30. The method of claim 27, further comprising performing automated digital microscopy (ADM) on the cells positive for pan-CK, EGFR, HER2, MUC1, or PLS3.

31. (canceled)

32. (canceled)

33. (canceled)

34. (canceled)

35. (canceled)

36. A method of detecting and treating non-small-cell lung cancer in a subject, the method comprising:
obtaining or having obtained nucleated cells from a subject;
contacting the nucleated cells with an anti-pan-cytokeratin (pan-CK) antibody, an anti-epidermal growth factor receptor (EGFR) antibody, an anti-human epidermal growth factor receptor 2 (HER2) antibody, an anti-mucin 1 (MUC1) antibody, an anti-plastin 3 (PLS3) antibody, and an anti-programmed death-ligand (PD-L1) antibody;
detecting binding of the anti-pan-CK antibody, the anti-EGFR antibody, the anti-HER2 antibody, the anti-MUC1 antibody, the anti-PLS3 antibody, and the anti-PD-L1 antibody to the nucleated cells, wherein binding of the anti-pan-CK antibody, the anti-EGFR antibody, the anti-HER2 antibody, the anti-MUC1 antibody, the anti-PLS3 antibody and the anti-PD-L1 antibody is to the nucleated cells, wherein a cell positive for pan-CK, EGFR, HER2, MUC1, PLS3, and PD-L1 indicates non-small-cell lung cancer in the subject; and
treating the non-small-cell lung cancer in the subject with a PD-L1 inhibitor.

37. (canceled)

38. (canceled)

39. (canceled)

40. (canceled)

41. (canceled)

42. (canceled)

43. The method of claim 36, wherein the detecting of the binding of the anti-pan-CK antibody, the anti-EGFR antibody, the anti-HER2 antibody, the anti-MUC1 antibody, the anti-PLS3 antibody, and the anti-PD-L1 antibody, and anti FAP antibody to the nucleated cells is conducted by scanning the slide using fiber-optic array scanning technology (FAST).

44. The method of claim 43, further comprising performing automated digital microscopy (ADM) on the cells positive for pan-CK, EGFR, HER2, MUC1, PLS3, FAP or PD-L1.

45. (canceled)

46. (canceled)

47. (canceled)

48. (canceled)
